(19) United States
(12) Patent Application Publication
(10) Pub. No.: US 2026/0047694 A1
(43) Pub. Date: Feb. 19, 2026

(54) INFANT CARRIER WITH PUMPING APPARATUS AND METHOD THEREOF

(71) Applicant: Connor Copeland, Lincoln, NE (US)

(72) Inventor: Connor Copeland, Lincoln, NE (US)

(21) Appl. No.: 19/304,585

(22) Filed: Aug. 19, 2025

Related U.S. Application Data
(60) Provisional application No. 63/684,549, filed on Aug. 19, 2024.

(51) Int. Cl. A47D 13/02; A61M 1/06

(52) U.S. Cl. CPC A47D 13/029; A61M 1/06

(57) ABSTRACT

An infant carrier and breast-feeding apparatus comprising a carrier portion and a pumping system. The carrier portion can include a first strap, a second strap, a waist strap, a first compartment formed on the waist trap portion. The pumping system can include having a first pump, a second pump, a first reservoir, a second reservoir, and at least one suction member couplable pumping apparatus. The apparatus facilitates a breastfeeding mother to be able to carry, pump and feed the infant while carrying on other activities.

### CROSS-REFERENCE TO RELATED APPLICATION

This U.S. patent application claims priority to U.S. Provisional Application 63/684,549 filed Aug. 19, 2024, the disclosure of which is considered part of the disclosure of this application and is hereby incorporated by reference in its entirety.

### FIELD OF THE INVENTION

The invention relates generally to an infant carrier having a couplable breastmilk pumping apparatus.

### BACKGROUND

Currently there are a number of solutions for collecting breastmilk. One of these solutions attempts to utilize a handheld pump, but this solution fails to meet the needs of the market because the user has to hold the pump while pumping, which may be very restricting to the user's movements. Another solution attempts to use a breast pump bra, but this solution is similarly unable to meet the needs of the market because the user is also limited to only certain movements and activities. Besides, the breast pump bra tends to provide a small storage capacity for the collected milk.

It would be advantageous to have an improved apparatus that can be wearable and configured to pump and store breastmilk on the go. Furthermore, it would also be advantageous to have an apparatus that allows the user to operate the apparatus hands-free. Still further, it would be advantageous to have an apparatus that can also function as an infant carrier. The apparatus of the present disclosure can fulfill an unmet need by providing an infant carrier having a couplable breastmilk pumping apparatus.

### BRIEF SUMMARY OF THE INVENTION

In one aspect, an infant carrier and pumping apparatus of the present disclosure can include a wearable garment configured to carry an infant, a couplable breast pump, a bottle, and an insulated holding tank configured to safely store breastmilk. The apparatus facilitates a breastfeeding mother to be able to carry, pump and feed the infant while carrying on other activities.

In another aspect, the present disclosure relates to an infant carrier and pumping apparatus, comprising pumping system and a carrier portion. The pumping system can include at least one pump. In some embodiments, the pumping system can include first pump, a second pump, a first reservoir, a second reservoir, and at least one suction member. The first pump can have first port and second port, and the second pump has first port and a second port. The first port of the first pump can be fluidly connected to at least one suction member, and the second port of the first pump is fluidly connected to the first reservoir. The first port of the second pump can be fluidly connected to the reservoir, and the second port of the second pump is fluidly coupled to the second reservoir. The carrier portion can include a first shoulder strap, a second shoulder strap, a waist strap, and a first compartment formed on the waist trap portion. The first compartment portion can be configured to house the first reservoir of the pumping system. The first pump can provide a breast pumping function carrying milk from the breast to the reservoir, while the second pump can provide a pumping of the stored milk in the reservoir to the bottle. The two pumps can operate individually and/or simultaneously.

The first compartment can be formed on a front side of the waist strap portion and can extend generally perpendicular from a first surface of waist strap portion and has a top side, a bottom side, a front side, a right side, a left side, and a back side, wherein the back side is part of the waist strap portion. The top side can provide a seating area or support for a child or infant. The top side has a padded portion. In some exemplary embodiments, the compartment can be insulated. The apparatus can include a first suction member and a second suction member. The apparatus can further include a connector clip portion having a first inlet and second inlet and an outlet. The first inlet is fluidly coupled to the first suction member, and the second inlet is fluidly coupled to the second suction member. The carrier portion can further comprise a coupling member on the first strap and second coupling member on the second strap, wherein the first coupling member can be removably coupled to a first corresponding coupling point and the second coupling member can be removably coupled to a second corresponding coupling point.

The apparatus can further include a third coupling point and a fourth coupling point, wherein the first coupling member can be removably coupled to the third coupling point and the second coupling member can be removably coupled to the fourth coupling point. A support portion can extend between the first strap and the second strap configured to support a child during use of the apparatus. A user can move the first strap from a first position at the first coupling point to a second position at the third coupling point. Similarly, a user can move the second strap from a first position at the second coupling point to a second position at the fourth coupling point. The outlet can be fluidly connected to the first port of the first pump. A cold pack can be included in the compartment portion to maintain the temperature of fluid in the reservoir. The first strap and second strap form a cross-configuration in the rear of the carrier portion. The second reservoir can have an inlet portion, a reservoir portion, and top portion, wherein the reservoir portion has a first opening and a second opening, wherein the inlet portion is removably coupled to the first opening and the top portion is removably couplable to the top portion. The second port of the second pump can be fluidly connected to the inlet portion of the second reservoir. The various fluid connections can be implemented using tubing between various components of the apparatus.

In another aspect, the disclosure relates to a method for pumping milk and providing milk to an infant simultaneously, comprising providing carrier and pumping apparatus of the present disclosure. Milk can be pumped from the user at the at least on suction member to the first pump and into the reservoir. The milk can then be pumped to a second pump and into a bottle apparatus.

The invention now will be described more fully hereinafter with reference to the accompanying drawings, which are intended to be read in conjunction with both this summary, the detailed description and any preferred and/or particular embodiments specifically discussed or otherwise disclosed. This invention may, however, be embodied in many different forms and should not be construed as limited to the embodiments set forth herein; rather, these embodiments are provided by way of illustration only and so that this disclosure will be thorough, complete and will fully convey the full scope of the invention to those skilled in the art.

### BRIEF DESCRIPTION OF THE DRAWINGS

FIG. 1A is a front view of an exemplary embodiment of the apparatus of the present disclosure;

FIG. 1B is a right-side view of an exemplary embodiment of the apparatus of the present disclosure;

FIG. 1C is a left side view of an exemplary embodiment of the apparatus of the present disclosure;

FIG. 1D is a top view of an exemplary embodiment of the apparatus of the present disclosure;

FIG. 1E is a perspective view of an exemplary embodiment of the apparatus of the present disclosure;

FIG. 1F is a side view of an exemplary embodiment of the apparatus of the present disclosure illustrating different coupling points of the straps;

FIG. 1G is a partial cutaway view of an interior cavity of a compartment portion of an exemplary embodiment of an apparatus of the present disclosure;

FIG. 2A is a front view of the of tank/pump of an exemplary embodiment of the apparatus of the present disclosure;

FIG. 2B is a back view of the tank/pump of an exemplary embodiment of the apparatus of the present disclosure.

FIG. 3A is a perspective view of an exemplary embodiment of the apparatus of the present disclosure;

FIG. 3B is a perspective view of an exemplary embodiment of the pumping system of the present disclosure;

FIG. 4A is a perspective view of an exemplary embodiment of a bottle assembly of the present disclosure; and

FIG. 4B is a cross-section view of an exemplary embodiment of a bottle assembly of the present disclosure; and

FIG. 5 is an illustration of a user wearing an exemplary embodiment of an apparatus of the present disclosure.

### DETAILED DESCRIPTION OF THE INVENTION

Following are more detailed descriptions of various related concepts related to, and embodiments of, methods and apparatus according to the present disclosure. It should be appreciated that various aspects of the subject matter introduced above and discussed in greater detail below may be implemented in any of numerous ways, as the subject matter is not limited to any particular manner of implementation. Examples of specific implementations and applications are provided primarily for illustrative purposes.

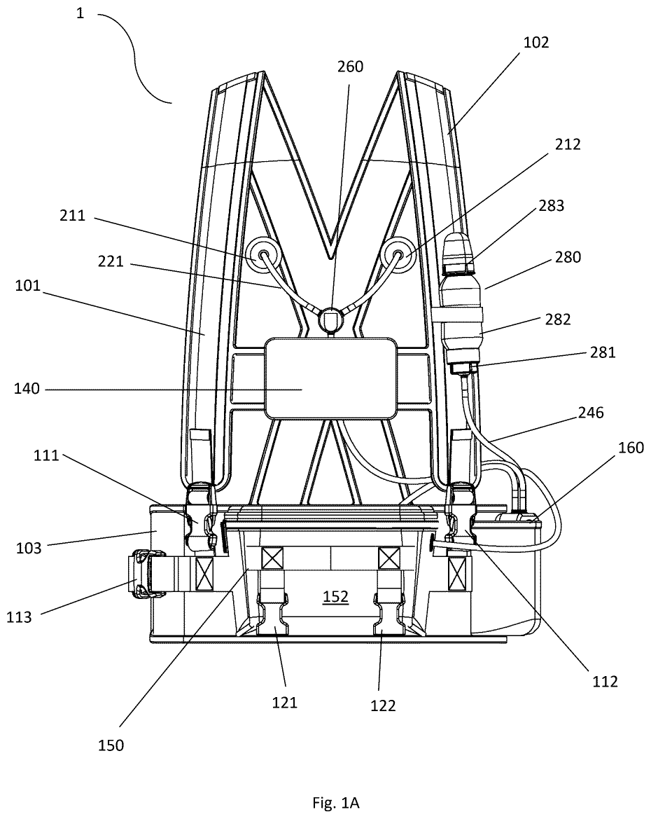

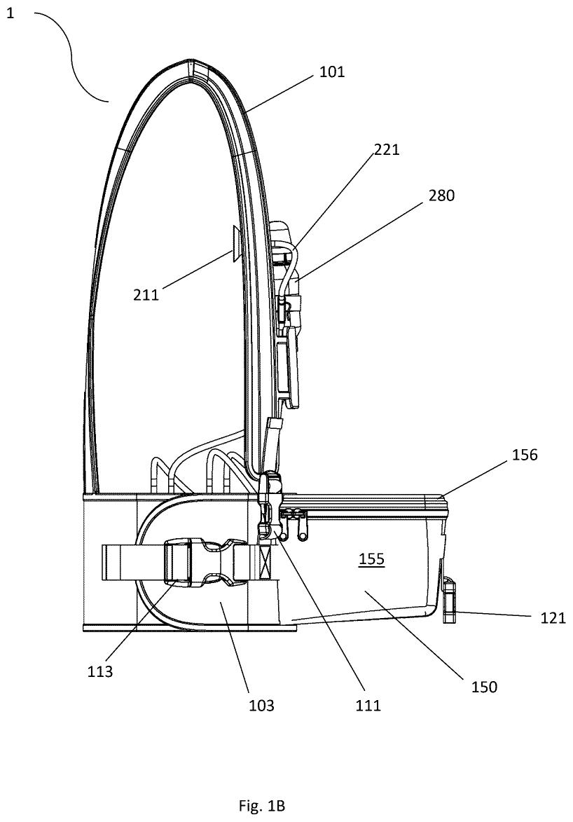

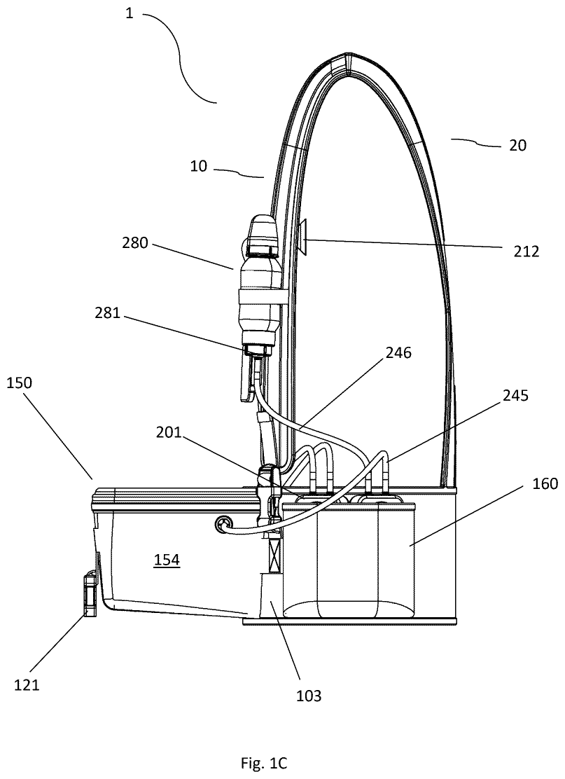

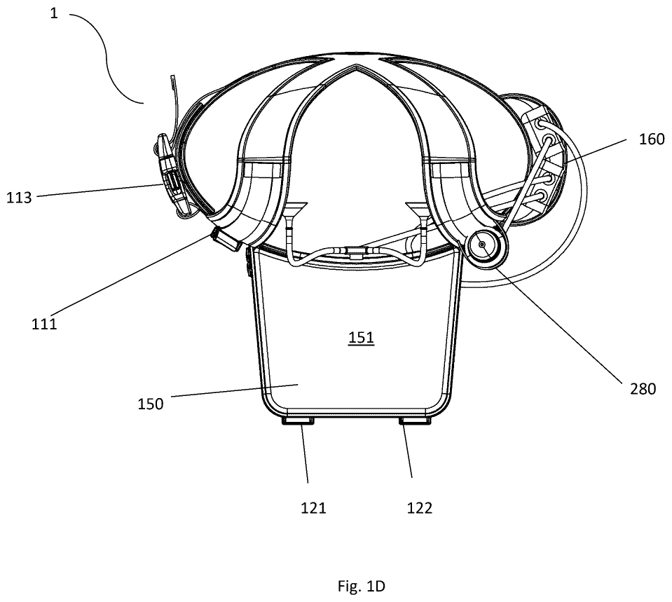

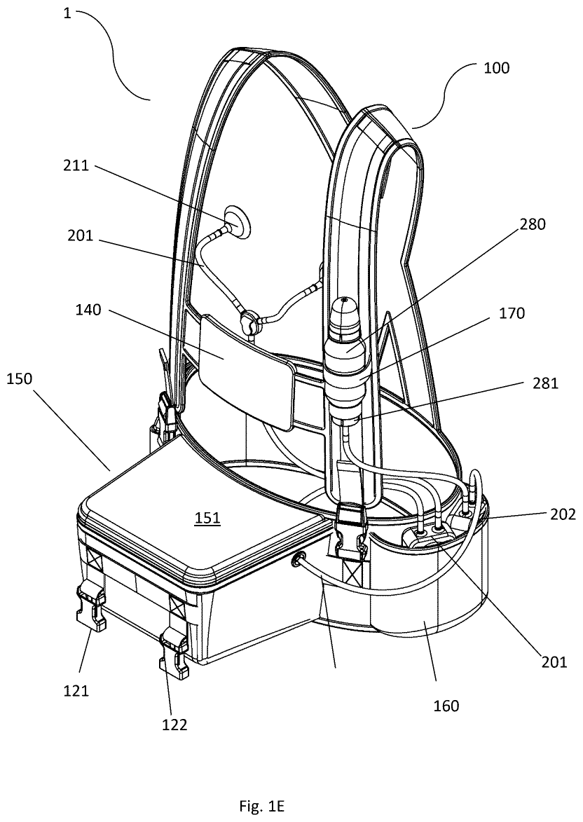

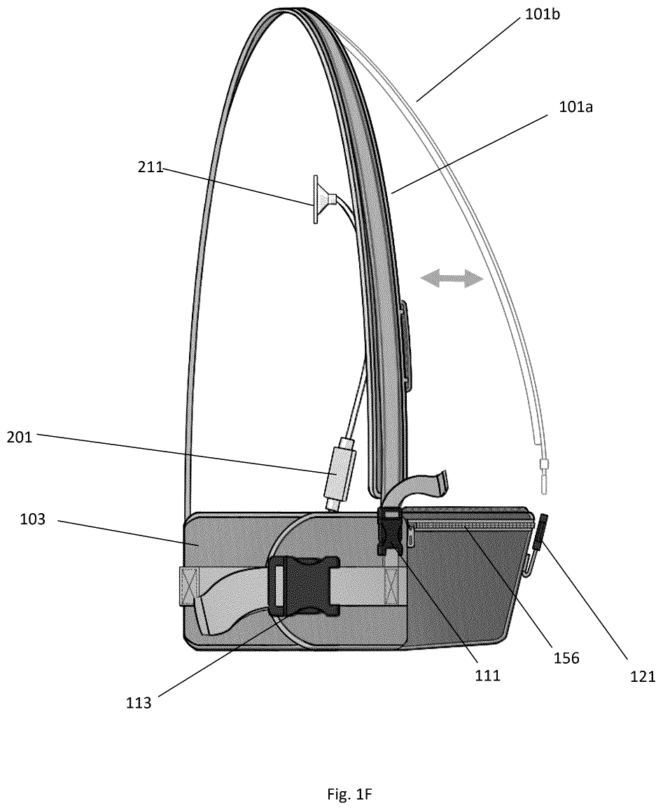

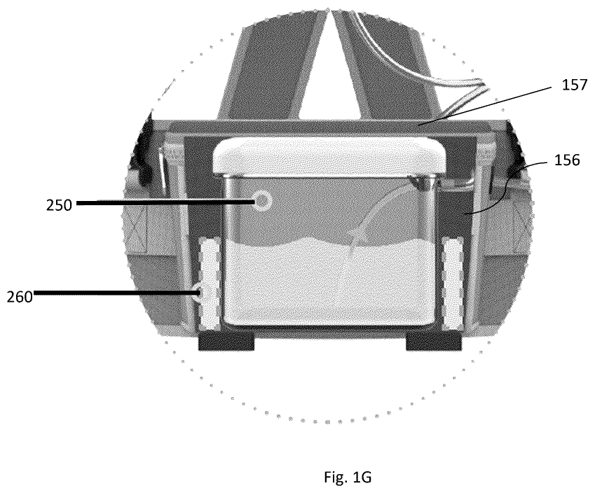

Referring now to FIGS. 1-5 of an infant carrier and pumping apparatus and system according to the present disclosure. The apparatus generally comprises a wearable carrier portion 100 configured to carry an infant and a pumping apparatus portion 200. The apparatus 1 can have a first side 10 and a second side 20. The carrier portion 100 can comprise at least a first strap 101, a second strap 102, and a waist band portion 103. The first strap 101 can be coupled to a first point on the waist band portion 103 in the second side and the second strap 102 can be coupled at the first point or another point along the second side 20 of the waist band portion 103. The first strap 101 can be removably coupled to a first coupling member 111 located on the first side 10 of the carrier portion 100. The strap coupling member 111, 112 can generally be located proximate to the waist band 103. Further reinforcement and/or adjustment straps can be provided to further support the waist band and apparatus 1 on a user 500. The second strap 102 can be removably coupled to a second coupling member 112 located on or proximate to the front side of the carrier portion as shown in FIG. 1A.

In some exemplary embodiments, the carrier portion 100 can be a wearable garment generally having at least two straps 101,102 configured to wrap around the shoulders and torso of a wearer, a padded waist belt 103 configured to wrap around a waist of the wearer, an infant padded seat located at a front surface of the belt and configured to be positioned at the front of the wearer close the waist height of the wearer. The straps 101,102 may have a crossed configuration at the back portion, such as a cross or x-orientation, to provide extra back support. A support pad may be coupled to a middle support portion 140 of the at least two straps at the front portion, to provide extra support and comfort. In some exemplary embodiments, the carrier portion 100 may be made of a lightweight fabric material having some added expandable fabric portions for extra comfort and adjustability. The straps may have coupling members, such as buckles or clips for coupling the straps onto the waist belt or to the infant seat for securing the infant/child 510.

The carrier portion 100 can further include a third coupling member 121 and a fourth coupling member 122. The third coupling member 121 and the fourth coupling member 122 can be coupled to a portion of the waistband portion 103. The strap members 101,102 can be movable between the first and second coupling members 111,112 and the third and fourth coupling members 121,122 as illustrated in FIG. 1F. This allows for the strap to be moved between a first position and a second position depending upon the user and if they are also wanting to hold a child simultaneously. In generally, a user may keep the strap in a first position (101a) using the first and second coupling members without a child on the apparatus and then move the straps to a second position (101b) with the third and fourth coupling members when being used with a child.

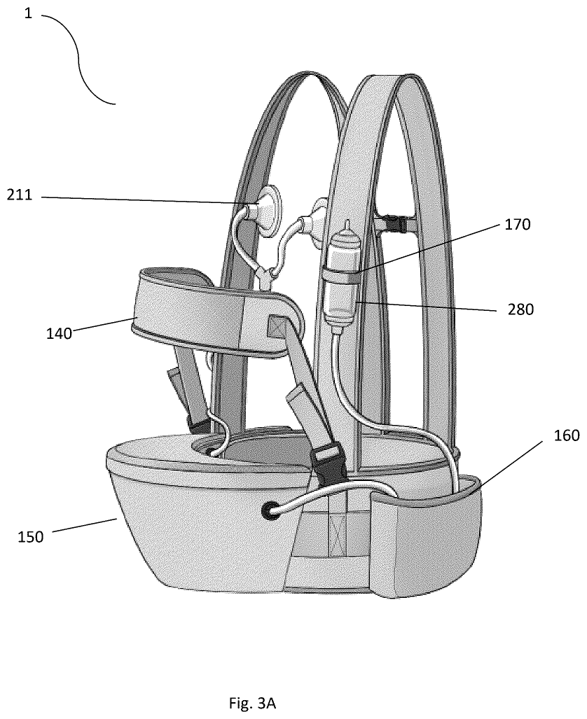

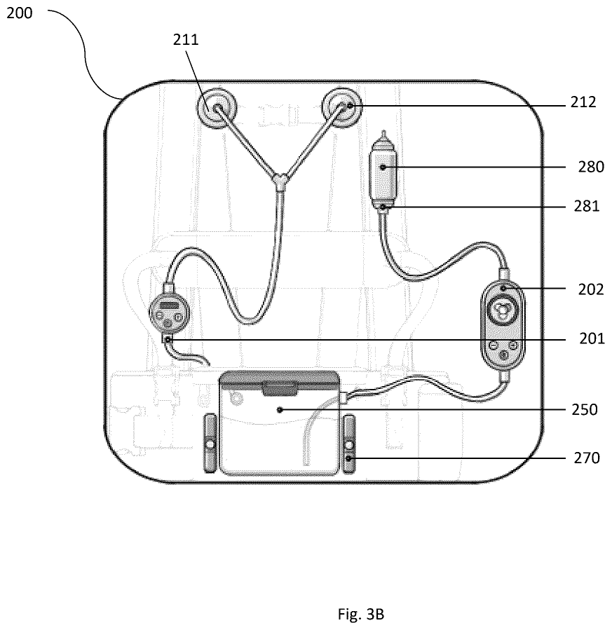

In some exemplary embodiments, a supporting strap or portion 140 can extend between the front side of the first strap 101 and the second strap 102. The support strap 140 can provide a back rest or additional support for a child or baby that is placed within the carrier portion 100 of the apparatus. This provides further comfort and safety for a user when the baby is being supported by the apparatus 1. In other exemplary embodiments, the support strap 140 can be removably couplable to a portion of the harness portion 100, including but not limited to the straps 101,012, or the waist strap 103 as shown in FIG. 3A. In some exemplary embodiments, the reservoir 250 can have a plurality of openings to fluidly connect the reservoir to the respective pumps.

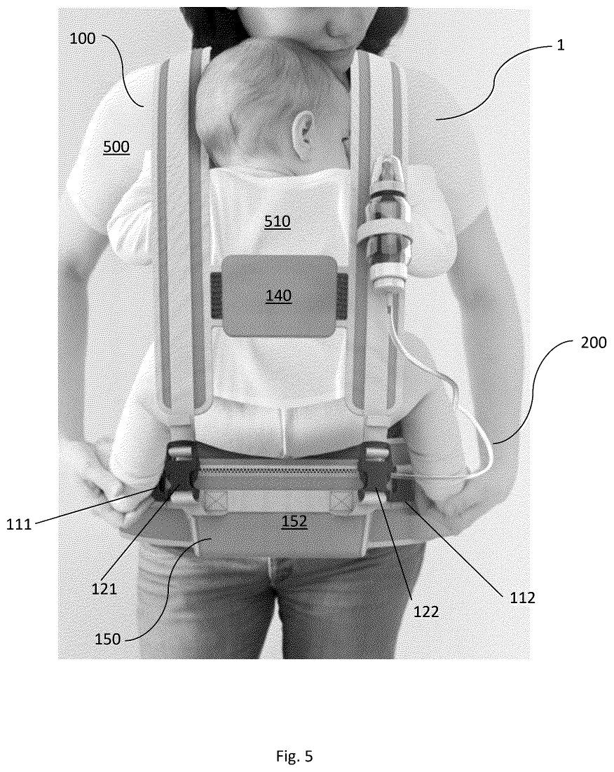

The waist band portion 103 can generally be positioned around the waist of a user proximate to a user's waist or hips as shown in FIG. 5. A coupling member 113 can be used to removably couple the coupling member 113. In some exemplary embodiments, the coupling members of the apparatus can take any suitable form including but not limited to snap members, hook loop fasteners or bands, buckles, g-hooks, d-rings, or clips. The clips can further be adjustable allowing for a user to easily tighten or loosen the straps 101,102 or waist band portion 103. In some exemplary embodiments, the coupling members can have a male and female portion to removably coupled the waist band or straps. The waist band portion 103 can further include a compartment 150 that can include a top side 151, front side 152, bottom side 153, and one or more left and right side 154,155. The compartment portion can extend generally perpendicularly from the side wall of the waist band portion 103 as shown in FIG. 1B-C. The first set of coupling member points 111, 112 can be located on or proximate to the waist band 103. A second set of coupling member points 121,122 can be located on or proximate to the front surface 152. This provides a user the ability to move the straps 101,012 from a first position at the first set of coupling points 111, 112 to a second position at the second set of coupling points 121,122.

FIG. 1G illustrates a cutaway of the front side of the 152 of the compartment portion 150. In some exemplary embodiments, the compartment portion 150 can include an interior cavity 156 configured to provide a storage area for a reservoir 250 or for the storage of various items. The cavity can have an interior volume. The compartment 150 can be openable through various sealing members 156 including fasteners, zippers or other means. The top side can open like a flap when the fastener is released. The compartment portion 150 can provide additional space for items such as a hot or cold pack 270 to help maintain the temperature within the compartment 150. The top side 151 of the compartment portion 150 can have a padded member 157 proximate or under the top surface 151 to provide a cushion for a child or user of the apparatus 1. The band portion 103 can further comprise additional pouch or storage portion 160 that can be used to house or store one or more components of the pumping portion or system 200. The storage portion can be used to store the one or more pumps. In some exemplary embodiments, a support member or strap 170 can be positioned or formed on one or more of the straps of the carrier portion as shown in FIG. 1E.

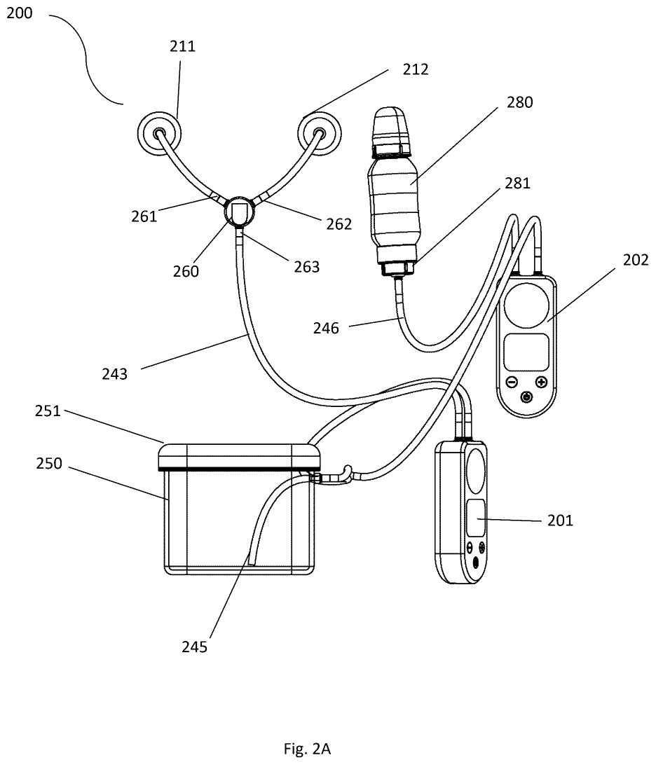

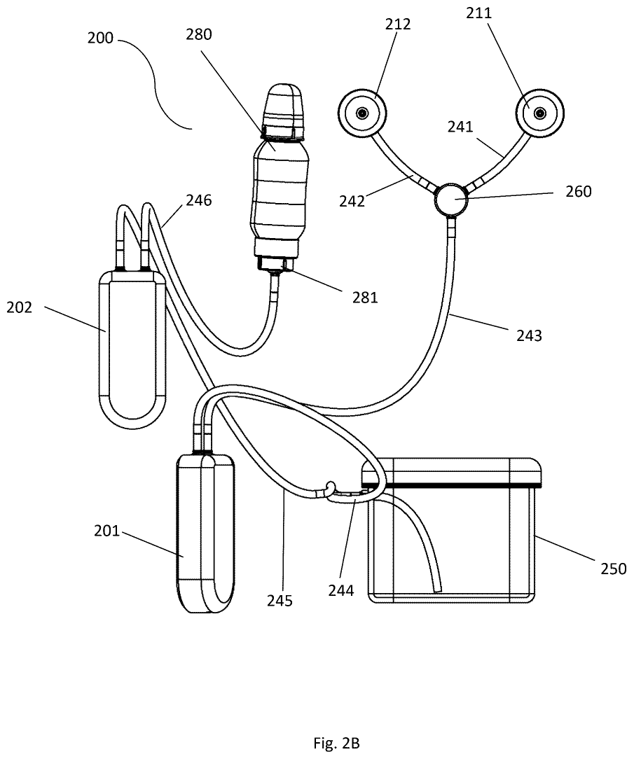

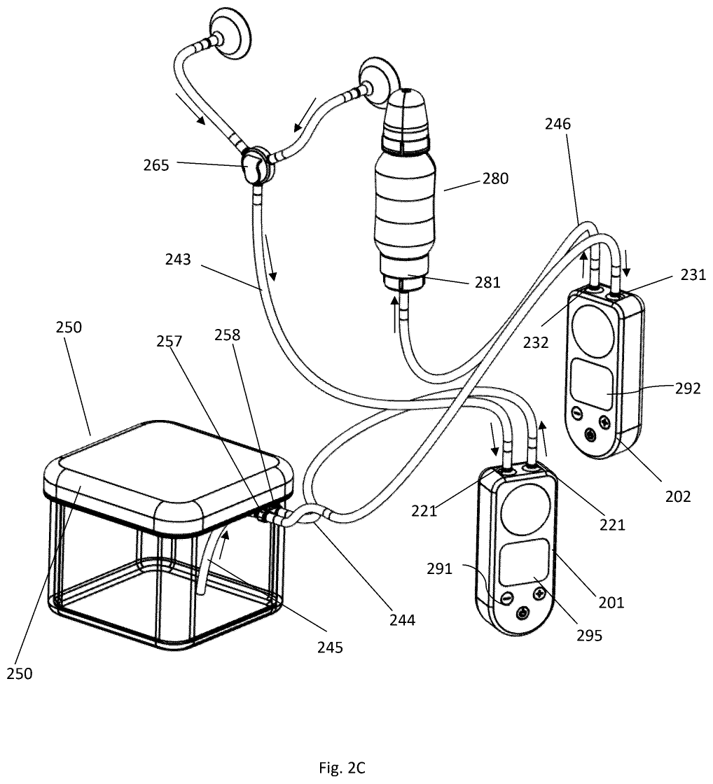

As shown in FIGS. 2A-C, a pumping system 200 of the apparatus 1 can include a first pump 201, a second pump 202, one or more suction cup members 211,212, a reservoir 250, a bottle member 280, and various tubing components 240 to fluidly couple or connect various components of the pumping system 200. In some exemplary embodiments, a first pump 201 can be a pump that can be coupled to the one or more suction cups members 211, 212 with a tubing member 243. The pumps of the apparatus can be any suitable pump. Some embodiments utilizing two suction cup members can include a connector clip 260 having a first inlet passage 261 and second inlet passage 262 and an outlet passage 263. The connector clip 260 can further include a clip portion 265 extending from a surface configured to clip to a shirt or garment of a user or a strap of the carrier portion 100. A first suction cup member 211 can be coupled to a first inlet 261 with a tubing member 241 and a second suction cup member 212 can be coupled to a second inlet 262 with a second tubing member 242. The outlet 243 can have a tubing member 243 connecting the connector clip 260 to a first port 221 on the first pump 201. A second port 222 on the first pump 202 can be fluidly connected to reservoir 250 with a tubing member 244. The pump 201 can pump breast milk from a user to the reservoir 250 while a user is wearing the apparatus 1. The milk or liquid can move from the one or more suction members to the reservoir 250. The reservoir can be removable from the compartment portion 150 of the

The reservoir 250 can store the milk for the user to store later or alternatively be pumped out of the reservoir 250 to a bottle 280 with a second pump 202. The second pump can be fluidly connected to the reservoir 250 with a tubing member 245 that can be fluidly connected to a first port 231 on the second pump 202. The reservoir can have one or more opening or ports 257 to provide a fluid connection to the tubing member. In some exemplary embodiments, a tubing member 245 can extend through the first port 257 of the reservoir and extend towards the bottom of the reservoir to allow a user to pump out milk or a liquid from the bottom of the reservoir 250. A second port or aperture 258 can allow a tubing member 244 to transport milk from the breast of a user to the reservoir 250. The reservoir 250 can have a removably couplable lid 259 to allow for easy cleaning of the reservoir 250.

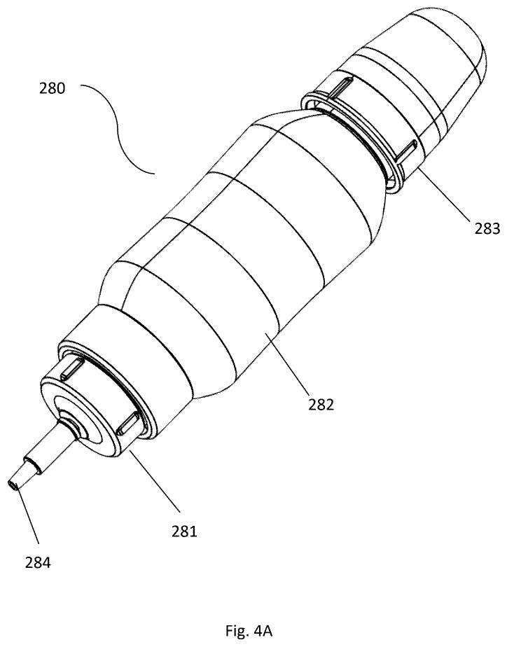

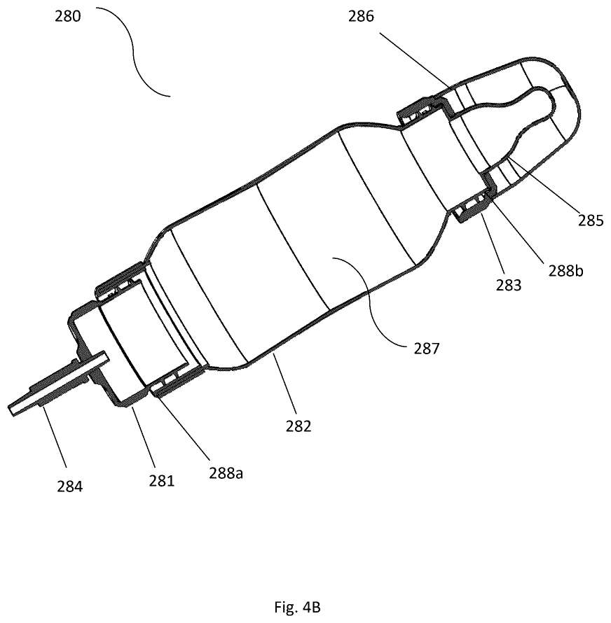

The pump 202 can be fluidly connected to a bottle member 280 with a tubing member 246 at a second port 232. A bottle member 280 can include various components including a base inlet portion 281, a reservoir portion 282, and a top portion 283 as shown in FIGS. 4A-B. The base inlet portion 281 and top portion 283 can be removably couplable to the bottom portion using any suitable means including threaded connectors or press fit. The inlet portion 281 can include first port 284 that can be coupled to a tubing member 246 to allow milk or a fluid to be pumped into the bottle 280. The top portion can include a nipple 285 and a cap portion 286. The bottle can have a cavity 287 for storing milk or a fluid pumped into the interior cavity that can be readily used by a user. The reservoir portion 282 can have an opening or aperture 288a,b formed on each end to accept the inlet portion 281 and the top portion 283. A separate bottom member can be used to fully enclose the bottle when the pump is not being used to allow a user to use the bottle to operate a standard bottle.

In some exemplary embodiments, it should be understood that a singular pump could potentially be used to first pump the milk into the reservoir and then to a bottle. The pump could have multiple ports that take the place of a two-pump system or alternatively tubing could be reorganized based upon a user's needs. In some exemplary embodiments, the pumps can be peristaltic pumps. The pumps can have one or more controls 291, 292 and be controlled on the face of the pumps. In some exemplary embodiments, the pumps can have one or more transceivers and be controlled over a wireless networks, including but not limited to a NFC, Wi-Fi, Bluetooth, or other networks. A wireless device such as a cell phone could be used as a controller to the pump(s). The controls can increase and decrease pumping power and also include a chargeable port to charge the controllers of pumps which can be powered by a power source, such as a battery. A display 295 on the pump can provide information to a user on the pump status.

The apparatus 1 of the present disclosure can allow a user to pump breast milk while simultaneously utilizing the milk for feeding a child through a bottle member 280. This provides a user flexibility to initially pump breast milk until the child needs to start feeding which can then be pumped into the bottle from the reservoir. The carrier portion 100 allows for the child to easily sit and be supported during feeding and/or pumping. As shown in FIG. 2C, the apparatus can pump milk out of the breast from the suction members to the first pump and into the reservoir. The milk can then be pumped from the reservoir to the second pump and to the bottle.

While the invention has been described above in terms of specific embodiments, it is to be understood that the invention is not limited to these disclosed embodiments. Upon reading the teachings of this disclosure many modifications and other embodiments of the invention will come to mind of those skilled in the art to which this invention pertains, and which are intended to be and are covered by both this disclosure and the appended claims. It is indeed intended that the scope of the invention should be determined by proper interpretation and construction of the appended claims and their legal equivalents, as understood by those of skill in the art relying upon the disclosure in this specification and the attached drawings.

What is claimed is:

1. An infant carrier and pumping apparatus, comprising:
pumping system comprising a first pump, a second pump, a first reservoir, a second reservoir, and at least one suction member,
wherein the first pump has first port and second port and the second pump has first port and a second port,
wherein the first port of the first pump is fluidly connected to at least one suction member and the second port of the first pump is fluidly connected to the first reservoir, and the first port of the second pump is fluidly connected to the reservoir and the second port of the second pump is fluidly coupled to the second reservoir; and
a carrier portion comprising a first strap, a second strap, a waist strap, a first compartment formed on the waist trap portion,
wherein the first compartment portion is configured to house the first reservoir of the pumping system.

2. The apparatus of claim 1, wherein the first compartment is formed on a front side of the waist strap portion, wherein the first compartment extends generally perpendicular from a first surface of waist strap portion and has a top side, a bottom side, a front side, a right side, a left side, and a back side, wherein the back side is part of the waist strap portion.

3. The apparatus of claim 2, wherein the top side has a padded portion.

4. The apparatus of claim 3, the at least one suction members includes a first suction member and a second suction member.

5. The apparatus of claim 4, further comprising a connector clip portion having a first inlet and second inlet and an outlet.

6. The apparatus of claim 5, wherein the first inlet is fluidly coupled to the first suction member, and the second inlet is fluidly coupled to the second suction member.

7. The apparatus of claim 6, wherein the carrier portion further comprise a coupling member on the first strap and second coupling member on the second strap, wherein the first coupling member can be removably coupled to a first corresponding coupling point and the second coupling member can be removably coupled to a second corresponding coupling point.

8. The apparatus of claim 7, further comprising a third coupling point and a fourth coupling point, wherein the first coupling member can be removably coupled to the third coupling point and the second coupling member can be removably coupled to the fourth coupling point.

9. The apparatus of claim 8, a support portion extending between the first strap and the second strap configured to support a child during use of the apparatus.

10. The apparatus of claim 9, wherein a user can move the first strap from a first position at the first coupling point to a second position at the third coupling point.

11. The apparatus of claim 10, wherein a user can move the second strap from a first position at the second coupling point to a second position at the fourth coupling point.

12. The apparatus of claim 11, wherein the outlet is fluidly connected to the first port of the first pump.

13. The apparatus of claim 12, further comprising a cold pack configured to maintain the temperature of fluid in the reservoir, wherein the cold pack is stored within the compartment.

14. The apparatus of claim 13, wherein the first strap and second strap form a cross-configuration in the rear of the carrier portion.

15. The apparatus of claim 14, wherein the second reservoir has an inlet portion, a reservoir portion, and top portion, wherein the reservoir portion has a first opening and a second opening, wherein the inlet portion is removably couple to the first opening and the top portion is removably couplable to the top portion.

16. The apparatus of claim 15, wherein the second port of the second pump is fluidly connected to the inlet portion of the second reservoir.

17. The apparatus of claim 16, wherein the fluid connections between components is through tubing members.

18. A method for pumping milk and providing milk to an infant simultaneously, comprising:
providing carrier and pumping apparatus comprising:
pumping system comprising a first pump, a second pump, a first reservoir, a second reservoir, and at least one suction member,
wherein the first pump has first port and second port, and the second pump has first port and a second port,
wherein the first port of the first pump is fluidly connected to at least one suction member and the second port of the first pump is fluidly connected to the first reservoir, and the first port of the second pump is fluidly connected to the reservoir and the second port of the second pump is fluidly coupled to the second reservoir; and
a carrier portion comprising a first strap, a second strap, a waist strap, a first compartment formed on the waist trap portion,
wherein the first compartment portion is configured to house the first reservoir of the pumping system;
pumping milk from the user at the at least on suction member to the first pump and into the reservoir;
pumping the milk from the reservoir to a second pump and into a bottle apparatus.
